(19) United States
(12) Patent Application Publication
(10) Pub. No.: US 2008/0234742 A1
(43) Pub. Date: Sep. 25, 2008

(54) Head Fixation Device

(76) Inventor: Jose Ludovico CASCARINO, Buenos Aires (AR)

Correspondence Address: NORRIS, MCLAUGHLIN & MARCUS, P.A., 875 THIRD AVE, 18TH FLOOR, NEW YORK, NY, 10022, US

(21) Appl. No.: 11/683,702

(22) Filed: Mar. 8, 2007

(51) Int. Cl. A61B 17/70; A61B 17/04

(52) U.S. Cl. 606/257; 606/246; 606/301

(57) ABSTRACT

An inner fixation system for implanting into the body of a patient and for providing the necessary support for the head of the patient afflicted by spine disorders, the system comprising a stem at each side of the head of the patient, wherein each stem is connected to the cranium and to the sternum of the patient.

### BACKGROUND OF THE INVENTION

1. Field of the Invention

The present invention relates to the medical field and particularly to systems and devices employed in the medical field to correct bone disorders or to aid a patient afflicted by bone disorders and, more particularly the invention refers to a device for stabilizing and supporting the head of a patient when the patient is afflicted by disorders in the spine and even more particularly when the cervical vertebrae are so affected that they do not provide, momentarily or permanently, the corresponding stability and support to the head of the patient.

2. Description of the Prior Art

It is well know that the spine provides the entire support for an individual, like the human being, that moves while keeping its vertical position. When one or more vertebrae of the spine are affected by any disorder in a degree that the same do not provide the continuity in the supporting function of the spine one or more vertebrae must be repaired or aids must be inserted in order to provide the vertebrae again with their supporting function.

When the afflicted vertebrae are those easily accessed either from the back of through the ventral parts of the patient for reparation or correction purposes, many fixation devices may be employed. Several fixation systems, either external or internal, are well known in the art and comprise screws and rods for providing the spine with the lost structural resistance. These systems generally comprise a plurality of screws for fixing to particular portions of the vertebrae with the screws having a distal threaded portion to be screwed into the bone mass of the vertebra and a head generally provided with gripping means for receiving and gripping one or more rods. The rods, generally circular cross-section rods, pass through all the heads of the screws that have already fixed to the vertebrae and, once in position through all the aligned screws, the rods are fixed by the gripping means. This assembly forms a resistant structure for assisting the spine in its supporting function or for directly providing the entire support for the trunk of the individual.

The above screw-and-rod systems are easily employed all along the spine, fixing and connecting the necessary involved vertebrae, provided that the afflicted vertebrae, as indicated above, are easily acceded for the installation of the system. However, there are situations in that the involved vertebrae are not accessible. The upper portion of the vertebral column is formed by the cervical vertebrae, namely vertebra C1, the closest one to the head of the individual, to vertebra C7 the most distal one from the head. If vertebrae C3-C3 are involved in some kind of disorders, the installation of conventional systems may be easily possible as indicated above. However, serious problems and difficulties arise when, within the afflicted vertebrae, vertebrae C1 and C2 are involved. Vertebrae C1 and C2 are so close to the head, hence to the cranium or skull and the brain, that manipulation of any medical system close to this vertebrae is extremely difficult, dangerous and mostly impossible in some situations.

When the cervical vertebrae are afflicted by any disorder such as a partial or almost complete deterioration of the bone mass due to a cancer for example, as well as due to fractures or other destructive pathologies, the head does not have the necessary support from the spine and the head tends to fall over the shoulders or chest. It is quite evident that an individual can not live under this conditions and an aid is necessary to provide the natural support. A conventional system, if not the only one well known, for keeping the head of a patient vertically supported, is an external system consisting of a metal ring or crown that is externally fixed around the head by the use of four screws that are fixed in the cranium wall. The crown is fixed to the upper ends of four vertical rods the lower ends of which are firmly connected to a semi-rigid or rigid jacket that is worn by the patient. The patient must use all this assembly permanently, it can not be removed except by the professional in particular safety conditions, therefore the patient must learn to live together with this jacket and system and all the living activities, such as taking a bath, and any hygiene related activity as well as sleeping, must be done while wearing the system. It is quite evident that, while the system may keep the head well supported, this fixation system is extremely uncomfortable for a normal life as well as may be dangerous for some activities and is socially traumatic. The patient must also carry a tool and must learn to make some periodical adjustments because the several bolts, screws and nuts use to become loose, and may be dislodged if not controlled, with the corresponding risks for the patient. In addition, the parts of the head wherein the screws supporting the crown enter the cranium must be hygienized and disinfected at least twice a day.

Considering the fixation systems that are commercially available and the drawbacks thereof it would be convenient to have a new non external system that provide the necessary support for the head of a patient with spine disorders wherein the system does not require of attention and control by the patient.

### SUMMARY OF THE INVENTION

It is therefore an object of the invention to provide a new non external fixation system that is implanted into the body of the patient and provides the necessary support for the head of a patient afflicted by spine disorders, particularly in the cervical sector of the spine.

It is still another object of the present invention to provide a new inner fixation device that is implanted under the skin and tissues of the patient, without the need of any adjustment, control and attention to be made by the patient.

It is a further object of the present invention to provide an inner fixation device for implanting in a patient afflicted by a spine disorder under which the spine of the patient does not provide support for the head of the patient, the inner fixation device comprising:

at least one stem means having at least one upper end for connecting to the head of the patient and at least one lower end for connecting to at least one chest bone of the patient, wherein the chest bone to which the stem means is connected it is preferably the sternum of the patient.

It is a further object of the present invention to provide an inner fixation system for implanting into the body of a patient and for providing the necessary support for the head of the patient afflicted by spine disorders, the system comprising a stem at each side of the head of the patient, wherein each stem is connected to the cranium and to the sternum of the patient.

It is a further object of the present invention to provide an inner fixation device for implanting in a patient afflicted by a spine disorder under which the spine of the patient does not provide support for the head of the patient, the inner fixation device comprising:

left stem means having at least one upper left end for connecting to a left side of the head of the patient and at least one lower left end for connecting to at least one chest bone of the patient, and

right stem means having at least one upper right end for connecting to a right side of the head of the patient and at least one lower right end for connecting to at least one chest bone of the patient, wherein the chest bone to which the left and right stem means are connected it is preferably the sternum of the patient.

The above and other objects, features and advantages of this invention will be better understood when taken in connection with the accompanying drawings and description.

### BRIEF DESCRIPTION OF THE DRAWINGS

The present invention is illustrated by way of example in the following drawings wherein:

FIG. 1 shows a side elevational view of a cranium and spine of a patient afflicted by a spine disorder wherein the cervical vertebrae are so dramatically affected that the spine is mostly collapsed and the head is fallen down over the shoulders as shown by the phantom line while the normal profile of the spine is indicated by a solid line;

FIG. 2 shows a side elevational view of the cranium and spine of FIG. 1 wherein the cranium has been lifted to its correct position by means of a conventional system comprising screws and rods, this system being used in combination with the conventional external crown;

FIG. 3 shows a side elevational view of the cranium and spine of FIGS. 1 and 2 wherein the cranium is kept in its normal and correct position by means of an inner fixation device according to a preferred embodiment of the invention;

FIG. 4 shows a front elevational view of the cranium, spine and the inventive fixation system as shown in FIG. 3;

FIG. 5 is a partial perspective view of an upper end of a stem and upper fixing means for connection to the cranium according to an embodiment of the invention, and

FIG. 6 is a front elevational view of lower ends of two stems and lower fixing means for connection to the sternum of the patient.

### DESCRIPTION OF THE PREFERRED EMBODIMENTS

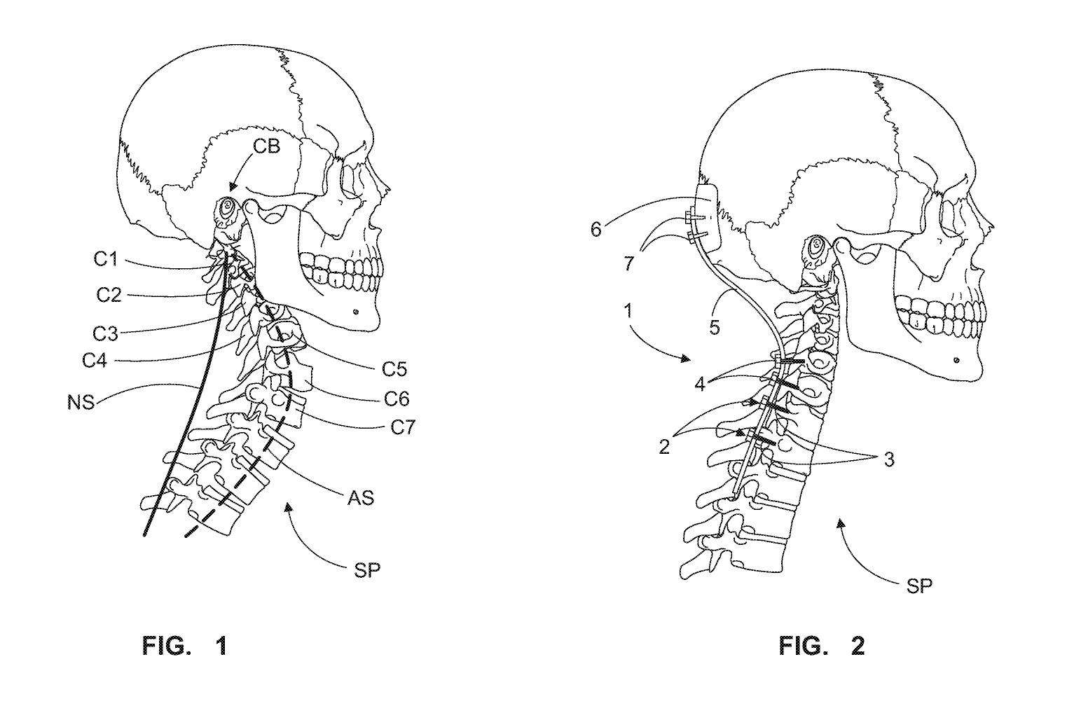

Now referring in detail to the drawings it may be seen from FIG. 1 the cranium and part of the spine of a patient afflicted by a disorder in the upper portion of the Spine. Cranium CR is connected at a base CB thereof to the cervical vertebrae schematically depicted and indicated by C1-C7. A normal profile of a spine in correct conditions must be extended more or less along solid line NS, however, under determined illness conditions such as a neck pathology, for example, the cervical vertebrae may be affected and some part o mostly part thereof may be deteriorated in such an extent that the spine keeps only parts of the original vertebrae. Under these conditions, the resistant structure of the spine is dramatically affected and, therefore, the spine SP, particularly at the cervical zone thereof, is incapable of resisting the weigh of the head and adopts a deformed, extremely curved or collapsed profile as shown along the phantom line AS.

When some lower cervical vertebrae are involved in the disease and the upper ones are healthy vertebrae, conventional systems comprised of screws and rods may be used as shown in FIG. 2, however this system is not enough to give the necessary support to the head in many of the afflictions. The system schematically shown in FIG. 2 and indicated by general reference 1 comprises a plurality of screws 2 firmly fixed into each one of the necessary vertebrae. Each screw, as it is well known, is comprised of threaded stem 3 for screwing into the bone mass of the vertebrae and corresponding heads 4 provided with some kind of retention, fastening or gripping means for permitting the pass of one or more rods 5 all along the aligned screws.

The rod or rods 5 are retained in each of the screws which in turn are retained into the bone of the vertebrae thus providing a resistant structure. To provide support to the head, a cranium plate 6 is screwed in the back of the cranium by use of corresponding screws 7 as illustrated. As indicated above, in many circumstances this system is insufficient to support the head and additional external systems are provided. One of them, perhaps the only one known, is the system known as “halo” or “crown” and comprises a metal ring arranged like a crown around the upper part of the head. About four screws are fixed, at respective distal ends thereof, to the ring, with respective proximal threaded ends of the screws being threaded into the cranium wall of the patient. The ring is also fixed to upper ends of about four rigid vertical rods the lower ends of which are firmly connected to a rigid jacket that must be worn by the patient permanently.

According to the invention, no external systems or devices are employed to provide a patient in need of a fixation device for the head with safety and better life quality. In effect, the inner fixation device according to a preferred embodiment of the invention, comprises at least one stem means, preferably a left stem 8 and a right stem 9, having at least one upper end for connecting to the head or cranium CR of the patient and at least one lower end for connecting to at least one chest bone, preferably the sternum ST of the patient. While the stems are connected to the sternum it is foreseen that, depending on the status of the sternum and other chest bones, the stems may be connected to any other bone capable of providing the necessary resistance to support the weight of the head.

Stems 8, 9 are connected at each side of the cranium and preferably at a portion thereof named parietal eminence, that is avascular and has proven to have the necessary wall thick and resistance for a connection according to the invention. Stems 8, 9 have the curvature that would be necessary to be accommodated internally and according to the anatomy of the neck and chest of the patient, therefore the illustrated curvatures are only exemplary curves.

Left stem 8 has an upper left end 10 for connection to upper left fixing means, preferably comprised of a left cranium plate 11, and right stem 9 has an upper right end 12 for connection to upper right fixing means, preferably comprised of a right cranium plate 13. Stem 8 has a at least one lower left end 14 and stem 9 includes at least one lower right end 15, wherein lower left and right ends are connected to respective lower left 16 and right 17 fixing means that preferably comprises only one chest plate 18 for fixing to any convenient chest bone but preferably for implanting in the sternum ST. At this stage of the description is clear that the weight of the head will be discharged along stems 8, 9 and supported by the sternum, or any other chest bone, through chest plate 18. This structure is a new concept that was never applied by implantation of an inner system according to the teachings of the invention. This system provides stabilization and support to the head of the patient without any external part being necessary. The inventive system may be combined or not with the occipital-cervical system illustrated in FIG. 2.

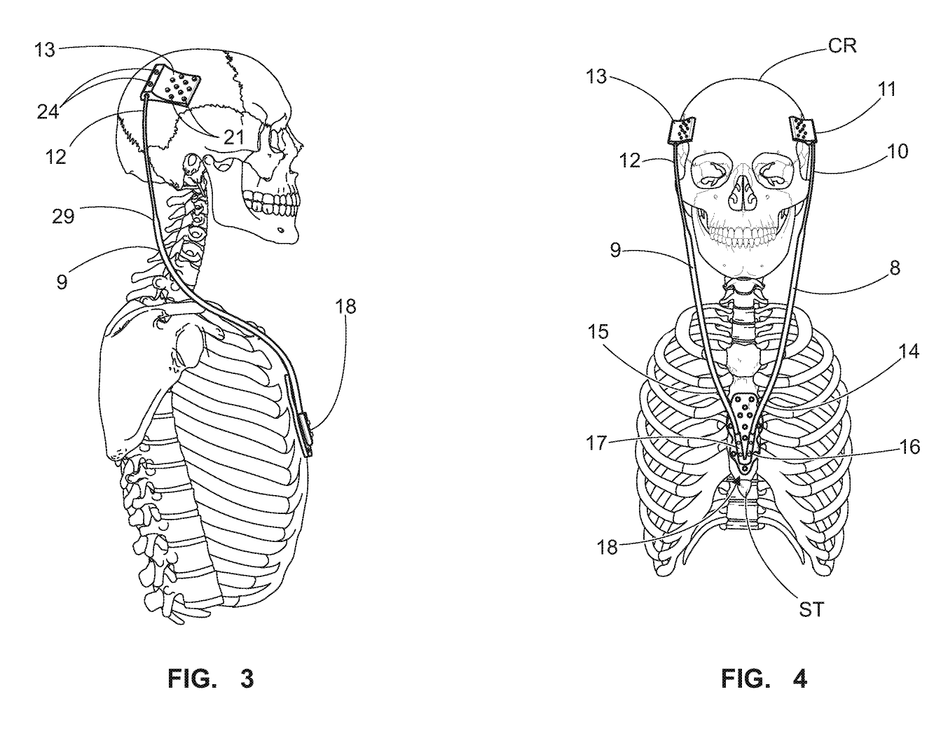

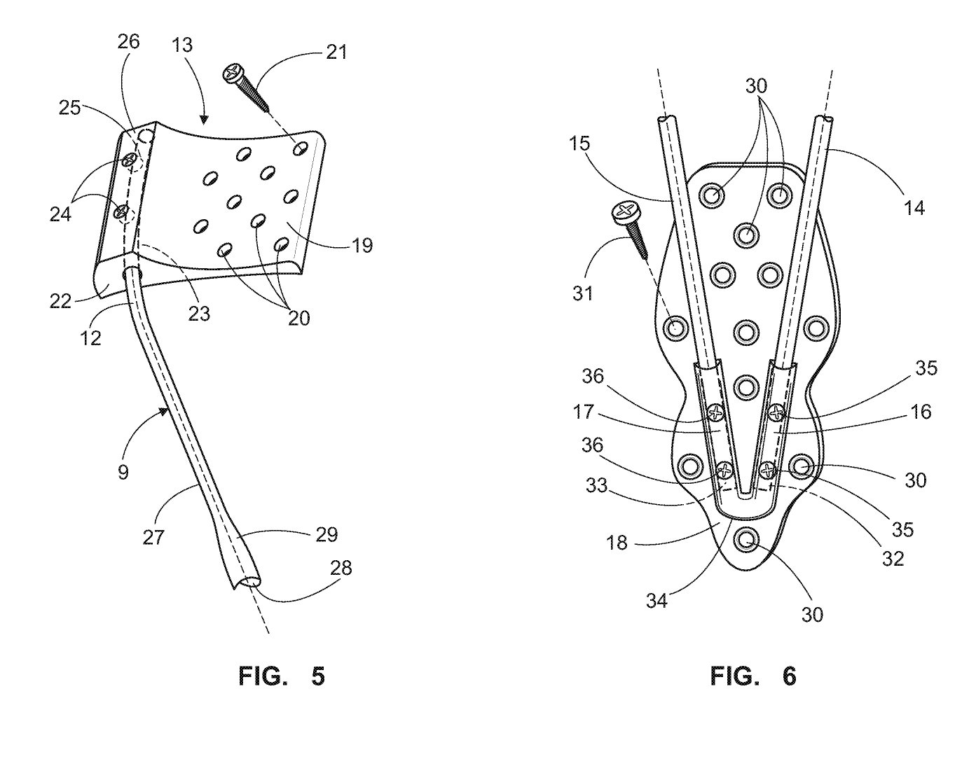

As better shown in FIG. 5, right cranium plate 13 is formed of a first slim section 19 having a plurality of orifices 20 for receiving screws 21, with only one shown for clarity purposes, for fixing to the cranium wall of the patient as shown in FIGS. 3, 4. Plate 13 also has a second thick section 22 including at least one cranium plate bore 23 for receiving said upper end 12 of the stem. Bore 23 may be aligned with section 22 or may be inclined, as shown, to better accommodate the stem when oriented towards the chest of the patient. Ticker section 22 includes fastening means, such as screws 24 which, threaded into corresponding fastening bores 25, permit to firmly and securely retain the upper end of stem 9 into bore 23. Bores 24 will be open at an inclined face 26 for facilitating installation of screws 24 into the bores. Ends 10, 12 may be provided with any aid, such as a flat surface or notch to enhance the retention of screws 24.

As clearly illustrated in FIG. 5, plate 13 may have any convenient shape and size capable of accommodating to the cranium anatomy but preferably it has a profile of smooth lines and curves. For example, while continuous lines are shown in FIG. 5, the periphery of plate 13 may be formed with lobules, as shown in FIG. 3, in order that each lobule extending out of the plate periphery includes an orifice 20. All these design questions are considered within the scope of protection of the invention. For optimizing weights and shapes stem 9 has a circular cross-section all along the stem but the section may vary according to the needs of the patient anatomy. In addition, the stem may have a small section 27 at an upper portion and a thicker section 28 at a lower portion of the stem, as illustrated in FIG. 5. The transition 29 between different sections 27, 28 will be as smooth as possible.

While only right plate 13 is illustrated in FIG. 5 it is clear that left plate 11 will have all the same characteristics and concepts of the invention with the proviso that the design will be inverted as compared to the left plate because of natural reasons. In other words, left plate 11, if illustrated like in FIG. 5, would be a mirror design of right plate 13.

Now, with reference to the lower ends 14, 15 of stems 8, 9, it is clear that end 14 enters and is retained into fixing means 16 and end 15 enters and is retained into fixing means 17. Means 16, 17 preferably form part of chest plate 18 which has a plurality of orifices 30 all appropriately arranged all along plate 18 for receiving screws 31, one in each orifice and only one illustrated for clarity purposes, for fixing the plate to sternum ST of the patient. Plate 18 includes at least one chest plate bore for receiving said at least one lower left end 14 and said at least one lower right end 15, but preferably plate 18 includes two chest bores, a left bore 32 and a right bore 33, indicated by phantom lines in FIG. 6, formed into respective fixing means 16, 17. Bores 32, 33 are arranged in a “V” pattern and join at the bottom thereof in a closed wall 34 that will merge the remaining of the plate in a smooth manner to prevent steps or sudden surface changes in the general design. Each bore 32, 33 is provided with fastening means, preferably screws 35, 36 fixed into corresponding chest plate bores not shown, for retaining said ends 14, 15 into the bores. Ends 14, 15 may be provided with any aid, such as a flat surface or notch to enhance the retention of screws 35, 36.

All the above described components may be manufactured of any of the well known bio medical materials such as stainless steel, titanium, nitinol, etc. but preferably titanium is employed. Furthermore, the shapes and cross-sections of any of the components will be designed in thicknesses and other size parameters that are the proper for the anatomy of the patient and body conditions.

While preferred embodiments of the present invention have been illustrated and described, it will be obvious to those skilled in the art that various changes and modifications may be made therein without departing from the scope of the invention as defined in the appended claims.

I claim:

1. An inner fixation device for implanting in a patient afflicted by a spine disorder under which the spine of the patient does not provide support for the head of the patient, the inner fixation device comprising:
at least one stem means having at least one upper end for connecting to the head of the patient and at least one lower end for connecting to at least one chest bone of the patient.

2. The device of claim 1, wherein the chest bone of the patient is a sternum.

3. The device of claim 1, wherein the at least one upper end of the stem means includes upper fixing means and the at least one lower end of the stem means includes lower fixing means.

4. The device of claim 3, wherein upper fixing means comprises a cranium plate having a plurality of orifices for receiving screws for fixing to a cranium wall of the patient.

5. The device of claim 4, wherein the cranium plate has a first slim section including said orifices and a second thick section including at least one cranium plate bore for receiving said at least one upper end of the stem means.

6. The device of claim 5, wherein said at least one cranium plate bore includes fastening means for retaining said at least one upper end of the stem means into the at least one cranium plate bore.

7. The device of claim 6, wherein the cranium plate has a curved profile for accommodating to the cranium curvature.

8. The device of claim 3, wherein lower fixing means comprises a chest plate having a plurality of orifices for receiving screws for fixing to a sternum of the patient.

9. The device of claim 8, wherein the chest plate includes at least one chest plate bore for receiving said at least one lower end of the stem means.

10. The device of claim 9, wherein said at least one chest plate bore includes fastening means for retaining said at least one lower end of the stem means into the at least one chest plate bore.

11. An inner fixation device for implanting in a patient afflicted by a spine disorder under which the spine of the patient does not provide support for the head of the patient, the inner fixation device comprising:
left stem means having at least one upper left end for connecting to a left side of the head of the patient and at least one lower left end for connecting to at least one chest bone of the patient, and
right stem means having at least one upper right end for connecting to a right side of the head of the patient and at least one lower right end for connecting to at least one chest bone of the patient.

12. The device of claim 11, wherein the chest bone of the patient is a sternum.

13. The device of claim 11, wherein the at least one upper left end of the left stem means includes upper left fixing means, the at least one upper right end of the right stem means includes upper right fixing means, the at least one lower left end of the left stem means includes lower left fixing means and the at least one lower right end of the right stem means includes lower right fixing means.

14. The device of claim 13, wherein the upper left fixing means comprises a left cranium plate and the upper right fixing means comprises a right cranium plate, the cranium plates having a plurality of orifices for receiving screws for fixing to a cranium wall of the patient.

15. The device of claim 14, wherein each cranium plate has a first slim section including said orifices and a second thick section including at least one cranium plate bore for receiving said at least one upper left and right ends of the stem means.

16. The device of claim 15, wherein said at least one cranium plate bore includes fastening means for retaining said at least one upper end of the stem means into the at least one cranium plate bore.

17. The device of claim 16, wherein each cranium plate has a curved profile for accommodating to the cranium curvature.

18. The device of claim 13, wherein the lower left fixing means and the lower right fixing means comprise a chest plate having a plurality of orifices for receiving screws for fixing to a sternum of the patient.

19. The device of claim 18, wherein the chest plate includes at least one chest plate bore for receiving said at least one lower left end of the left stem means and said at least one lower right end of the right stem means.

20. The device of claim 19, wherein said at least one chest plate bore includes fastening means for retaining said at least one lower left end of the left stem means and said at least one lower right end of the right stem means into the at least one chest plate bore.

21. The device of claim 20, wherein said at least one chest plate bore comprises two chest bores, a left bore and a right bore, arranged in a “V” pattern, with left bore for receiving the at least one lower left end of the left stem means and the right bore for receiving the at least one lower right end of the right stem means.

22. The device of claim 1, wherein the at least one stem means comprises a stem having a circular cross-section and made of titanium.

23. The device of claim 22, wherein the cross-section of the stem is variable along the length of the stem.

24. The device of claim 11, wherein the at least one left stem means comprises a left titanium-made stem having a circular cross-section, and the at least one right stem means comprises a right titanium-made stem having a circular cross-section.

25. The device of claim 24, wherein the cross-section of the left and right stems is variable along the length of the stems.
